(19) United States
(12) Patent Application Publication
(10) Pub. No.: US 2025/0149141 A1
(43) Pub. Date: May 8, 2025

(54) OPTIMIZING THERAPEUTICAL INTERACTIONS

(71) Applicant: KONINKLIJKE PHILIPS N.V., EINDHOVEN (NL)

(72) Inventors: Murtaza Bulut, Eindhoven (NL); Ron Martinus Laurentius van Lieshout, Eindhoven (NL); Mark Jacobus Rosalie Joseph Bouts, Cambridge, MA (US); Laurentia Johanna Huijbregts, Eindhoven (NL)

(21) Appl. No.: 18/661,864

(22) Filed: May 13, 2024

Related U.S. Application Data
(60) Provisional application No. 63/465,931, filed on May 12, 2023.

(51) Int. Cl. G16H 20/10; G16H 10/60; G16H 50/30

(52) U.S. Cl. CPC G16H 20/10; G16H 10/60; G16H 50/30

(57) ABSTRACT

A system for optimizing therapeutical treatments includes a central computer with a memory that stores instructions; and a processor that executes the instructions. When executed by the processor, the instructions cause the system to: receive at least one of an identification of a pharmaceutical therapeutic for reaching a first healthcare goal or an identification of a digital therapeutic for reaching the first healthcare goal; retrieve interaction information relating to potential for interactions for the first healthcare goal; determine that a potential interaction exists between the pharmaceutical therapeutic and the digital therapeutic with respect to the first healthcare goal based on the interaction information for the first healthcare goal; and generate feedback to reach the first healthcare goal.

### CROSS-REFERENCE TO RELATED APPLICATIONS

This patent application claims the priority benefit under 35 U.S.C. § 119(e) of U.S. Provisional Application No. 63/465,931, filed on May 12, 2023, the contents of which are herein incorporated by reference.

### BACKGROUND

Digital therapeutics (DTx) is an emerging field in healthcare, and involves integrating a subgroup of digital health applications into healthcare. DTx refers to software tested and validated through randomized clinical trial and approved or otherwise evaluated by regulatory bodies such as the United States Food and Drug Administration (USFDA). DTx can aim to prevent, manage, or treat a medical disorder or disease. DTx may produce a validated medical intervention which may be driven and delivered via a digital health application, and which may be complemented by hardware, medical device, service or medication. Similar to traditional pharmaceutical therapeutics (Tx), some instances of DTx are available only through prescription by healthcare providers, while other DTx can be obtained without a prescription. Due to the introduction of recent regulations in some jurisdictions, some types of DTx may be eligible for reimbursements, may be subject to various approval options, and/or may be treated as medical devices.

Online application stores have many lifestyle support applications that promote exercise, sleep, nutrition habits, psychological therapy and so on. Currently, approximately two thirds (⅔) of the subgroup of digital health applications characterizable as DTx are for treatment of neurological problems such as attention deficit hyperactivity disorder (ADHD), autism spectrum disorder (ASD), schizophrenia, depression, and bipolar disorders. However, the range of conditions addressable by DTx is expanding, with possibilities of DTx being used for gastrointestinal, cardiovascular, diabetes and sleep related applications. Other digital health applications include solutions for monitoring pharmaceutical regimen parameters including intake time and dosage with the purpose of precisely determining dosing.

A typical DTx will include at least two instances, one instance facing the patient and the other instance facing the clinician. The instances are provided via software and a user interface, such as applications and/or websites used by a smartphone or computer. The patient-facing instance is typically provided in a form of an interactive smartphone application. The clinician-facing instance may be provided via a dashboard that provides progress monitoring and guidance for patient activity.

A growing problem in the healthcare field is the growing number of digital health applications, primarily due to the shorter and easier path to development and approval compared to pharmaceutical therapeutics. The growing number of digital health applications presents a problem of specifically selecting the best application for a patient. The current approach is database based, where a list of suggested application(s) per condition is maintained by regulating bodies that approve applications. This is still possible only because there are limited numbers of prescription digital therapeutics (PDTs), but this approach may not be sustainable for much longer.

DTx offer a new dimension to disease management, increasing the options for both clinicians and patients. DTx may also increase the potential for interactions with pharmaceutical therapeutics. Currently, pharmaceutical-to-pharmaceutical interactions are resolved by a database that tracks and shows potential interactions to clinicians. However, a database approach is not particularly appropriate for DTx interactions with pharmaceutical interactions given the dynamic nature of applications. Applications regularly receive new updates where functionality of existing features may change, new features may be added, and current features may be removed. A classical database approach is not appropriate for keeping track of these changes and the resultant effects on interactions with pharmaceutical therapeutics. Additionally, the clinical effect and benefits of interactions between DTx and pharmaceutical therapeutics may be more difficult to track and quantify than pharmaceutical therapeutics due to the effects of DTx being incremental and spread over longer periods of time. This is because of high variability in how DTx are currently used by different patients or clinicians, and less rigorous development, test, and approval procedures.

As the healthcare field transitions to using both DTx and pharmaceutical therapeutics to reach disease management goals, avoidance of harmful interactions will be a concern. Such interactions can cause confusion and prevent clinicians from tracking and adjusting the disease management, and can create false expectation in patients resulting in non-optimal adherence to disease management plans. To help patients and clinicians achieve intended disease management goals in a traceable and controlled manner, there is a need for a solution to address the potential for interactions between DTx and pharmaceutical therapeutics, or between two forms of DTx. This requires solutions helping clinicians to select pharmaceutical therapeutics and DTx in an informed manner at the beginning of the treatment and helping patient and clinicians track effects and manage the potential interactions throughout. There is a need for new solutions that continuously monitor interactions between therapeutics including DTx and use this information to improve selection and usage of new digital health applications and pharmaceutical therapeutics, as well as monitoring of these therapeutics once they are prescribed.

### SUMMARY

According to an aspect of the present disclosure, a system for optimizing therapeutical treatments includes a central computer with a memory that stores instructions; and a processor that executes the instructions. When executed by the processor, the instructions cause the system to: receive at least one of an identification of a pharmaceutical therapeutic for reaching a first healthcare goal or an identification of a digital therapeutic for reaching the first healthcare goal; retrieve interaction information relating to potential for interactions for the first healthcare goal; determine that a potential interaction exists between the pharmaceutical therapeutic and the digital therapeutic with respect to the first healthcare goal based on the interaction information for the first healthcare goal; and generate feedback to reach the first healthcare goal.

According to another aspect of the present disclosure, a method for optimizing therapeutical treatments includes receiving, at a central computer with a memory that stores instructions and a processor that executes the instructions, at least one of an identification of a pharmaceutical therapeutic for a first healthcare goal or an identification of a digital therapeutic for the first healthcare goal; retrieving, by the central computer, interaction information relating to potential for interactions for the first healthcare goal; determining that a potential interaction exists between the pharmaceutical therapeutic and the digital therapeutic with respect to the first healthcare goal based on the interaction information for the first healthcare goal; and generating feedback to reach the first healthcare goal.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for optimizing therapeutical interactions, in accordance with a representative embodiment.

FIG. 2 illustrates a method for optimizing therapeutical interactions, in accordance with a representative embodiment.

FIG. 3 illustrates a workflow for optimizing therapeutical interactions, in accordance with a representative embodiment.

FIG. 4 illustrates another workflow for optimizing therapeutical interactions, in accordance with a representative embodiment.

FIG. 5 illustrates another workflow for optimizing therapeutical interactions, in accordance with a representative embodiment.

FIG. 6 illustrates another workflow for optimizing therapeutical interactions, in accordance with a representative embodiment.

FIG. 7 illustrates another workflow for optimizing therapeutical interactions, in accordance with a representative embodiment.

FIG. 8 illustrates a system flow for optimizing therapeutical interactions, in accordance with a representative embodiment.

FIG. 9 illustrates another workflow for optimizing therapeutical interactions, in accordance with a representative embodiment.

FIG. 10 illustrates another workflow for optimizing therapeutical interactions, in accordance with a representative embodiment.

FIG. 11 illustrates a computer system, on which a method for optimizing therapeutical interactions is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, patients may be provided with solutions to optimize potential interactions between different types of therapeutics ranging from pharmaceutical therapeutics to DTx or between different DTx instances with different healthcare goals. The optimization of potential interactions may be important for patients with comorbidities, or patients using pharmaceuticals with narrow therapeutic intervals, pharmaceuticals with many interactions, or pharmaceuticals with many side effects. As a result, conflicting treatment goals may be managed for patients. The solutions described herein provide for selection and monitoring of therapeutics with a potential for interaction with one or more instances of DTx, so that negative interactions are minimized and positive interactions are maximized. Different combinations of therapeutics for a condition may be scored based on measured or expected effects and interactions, so that optimal combinations can be selected and then monitored once prescribed.

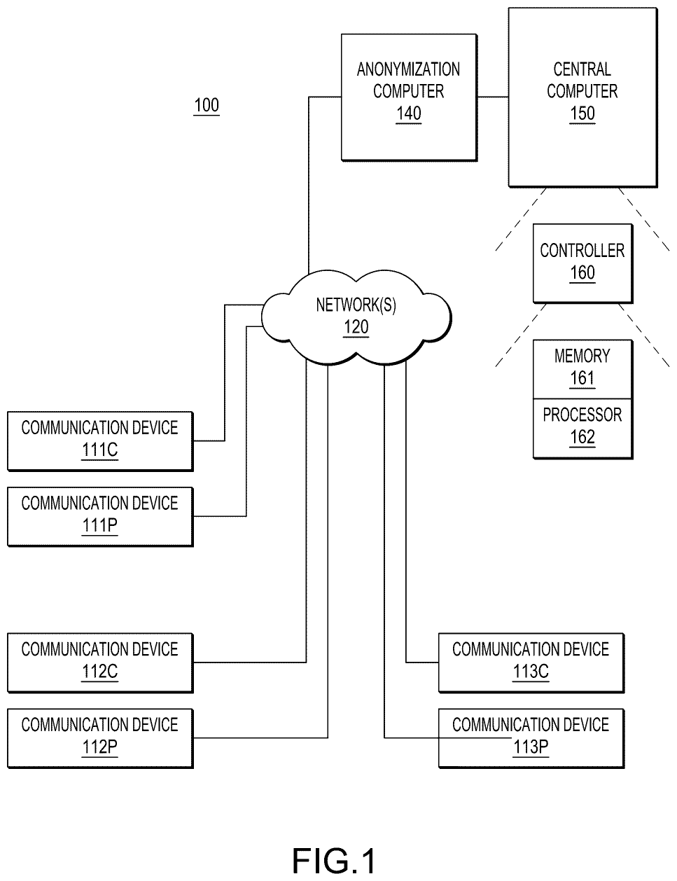

FIG. 1 illustrates a system 100 for optimizing therapeutical interactions, in accordance with a representative embodiment.

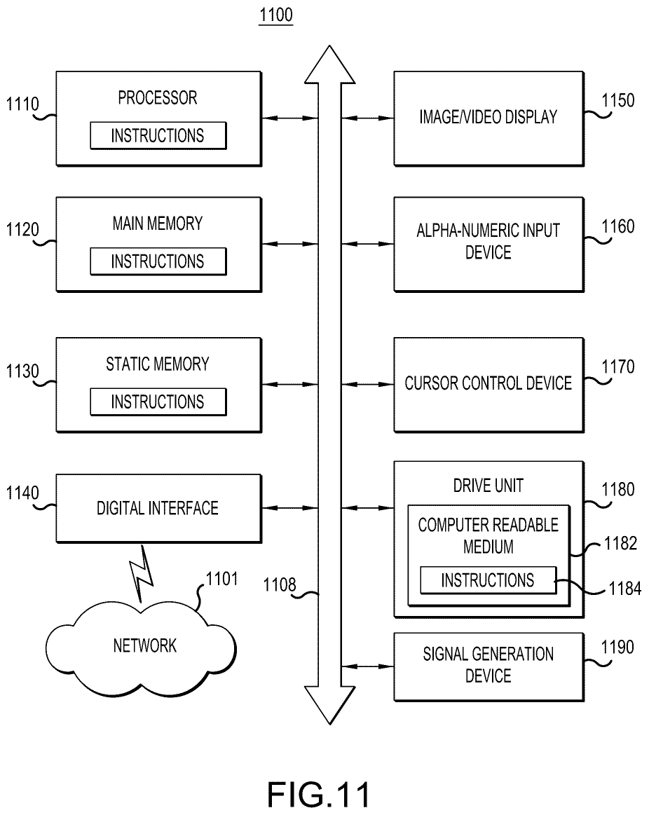

The system 100 in FIG. 1 is a system for optimizing therapeutical interactions and includes components that are distributed. The system 100 includes a first clinician communication device 111C, a first patient communication device 111P, a second clinician communication device 112C, a second patient communication device 112P, a third clinician communication device 113C, a third patient communication device 113P, one or more network(s) 120, an anonymization computer 140, and a central computer 150. The central computer 150 includes a controller 160, and the controller 160 includes a memory 161 and a processor 162. The memory 161 stores instructions and the processor 162 executes the instructions. A computer that can be used to implement the central computer 150 is depicted in FIG. 11, though a central computer 150 may include more or fewer elements than depicted in FIG. 1 or FIG. 11.

In some embodiments, the anonymization computer 140 and the central computer 150 are implemented in the cloud, such as by servers in a data center. The anonymization computer 140 is configured to anonymize data to be received at the central computer 150, and then provide the anonymized data to the central computer 150. For example, the anonymization computer 140 and the central computer 150 may perform a central service for one or more distributed medical groups and/or distributed medical facilities.

The controller 160 may perform some of the operations described herein directly and may implement other operations described herein indirectly. For example, the controller 160 may indirectly control operations such as by generating and transmitting content to be displayed on a display of the first clinician communication device 111C, the second clinician communication device 112C and/or the third clinician communication device 113C. The controller 160 may directly control other operations such as logical operations performed by the processor 162 executing instructions from the memory 161 based on input received from the patient communication devices, the clinician communication devices and/or users via other input mechanisms. Accordingly, the processes implemented by the controller 160 when the processor 162 executes instructions from the memory 161 may include steps not directly performed by the controller 160. An example of the content to be displayed on any one or more of the first clinician communication device 111C, the second clinician communication device 112C and/or the third clinician communication device 113C includes information such as instructions, scores, recommendations, and interaction information used by the central computer 150 to reach recommendations. The clinicians using the various clinician communication devices may be provided options displayed on the clinician communication devices, and may be provided an ability to select an option deemed to be most appropriate based on the operations of the central computer 150.

For the reasons described in the background, avoidance of mismatches will be important because pharmaceutical therapeutics are tested with limited numbers of people, and this will also be true of DTx. Moreover, considering that pharmaceutical therapeutics are developed, tested, and approved based on data from limited numbers of people, different than the people used for developing, testing, and approval of DTx, mismatches are to be expected. Additional complexities result from the uniqueness of every patient, which means that effects of pharmaceutical therapeutics and DTx have certain degrees of uncertainty and unpredictability for each individual. As a result, even after a combination of types of therapeutics is recommended or approved, the system 100 may monitor a patient for complications and compliance with expected behaviors via feedback from a DTx installed on any of the first patient communication device 111P, the second patient communication device 112P or the third patient communication device 113P.

As set forth above, the central computer 150 includes a controller 160 with a memory 161 and a processor 162. The system 100 that includes the central computer 150 may be provided for optimizing therapeutical treatments. When executed by the processor 162, the instructions may cause the system 100 to: receive at least one of an identification of a pharmaceutical therapeutic for reaching a first healthcare goal or an identification of a digital therapeutic for reaching the first healthcare goal; retrieve interaction information relating to potential for interactions for the first healthcare goal; determine that a potential interaction exists between the pharmaceutical therapeutic and the digital therapeutic with respect to the first healthcare goal based on the interaction information for the first healthcare goal; and generate feedback to reach the first healthcare goal. The feedback may be provided to a clinician communication device to provide information and, sometimes, selectable options.

As set forth above, the system 100 is provided so that potential interactions may be optimized for patients trying to reach one or more healthcare goals for which combinations of therapeutics are being considered. A healthcare goal may be a therapeutical goal, but may also include other types of goals such as sleep improvement, activity coaching and more. Additionally, the interaction information used to optimize the potential interactions does not necessarily refer to certainties in terms of interactions, and instead may include potential interactions which are not particularly certain, such as for a first diagnosed condition. An example of an uncertainty which is raised by interaction information may be a question or warning relating to simultaneous use of a DTx and a drug, and another DTx and another drug, or questions or warning relating to potential interactions for a first diagnosed condition. Moreover, warnings about potential interactions may include warnings about multiple certain or potential interactions.

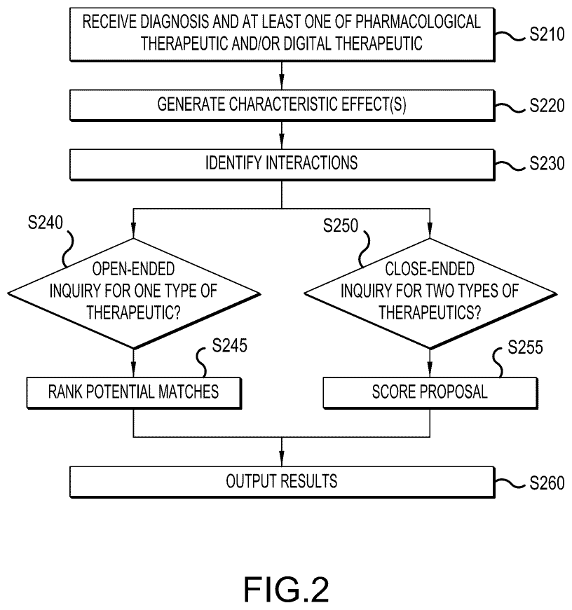

FIG. 2 illustrates a method for optimizing therapeutical interactions, in accordance with a representative embodiment.

The method of FIG. 2 may be performed by the system 100 including the central computer 150 with the controller 160.

At S210, a diagnosis and an identification of at least one of a pharmaceutical therapeutic and/or a digital therapeutic are received. For example, the computer 150 may receive the diagnosis and one or both of a pharmaceutical therapeutic and a digital therapeutic from the first clinician communication device 111C, the second clinician communication device 112C or the third clinician communication device 113C. The pharmaceutical therapeutic and the digital therapeutic may each be candidates for reaching one or more healthcare goal(s). In some embodiments, rather than a combination of a pharmaceutical therapeutic and a digital therapeutic, the identifications received at S210 may be for two or more digital therapeutics, or even more than two therapeutics such as a pharmaceutical therapeutic and a digital therapeutic. In some embodiments, the identification received at S210 may be received from either or both of a device used by a clinician such as a clinician communication device or a patient proposing a resolution for reaching a healthcare goal such as from a patient communication device.

At S220, one or more characteristic effect(s) are generated. The characteristics effect(s) are generated based on the received diagnosis and the one or more received therapeutics. The characteristic(s) may be combined with health information of the patient in addition to the diagnosis. Other types of information of the patient such as demographic information may also be received and used to generate the characteristic effect(s) at S220. As a result of generating the characteristic effect(s), the central computer 150 may retrieve interaction information relating to potential for interactions for the one or more healthcare goal(s). In some embodiments, rather than generating the characteristic effect(s) at S220, the characteristic effect(s) and the interaction information may both be retrieved, such as from a structured database with tables of information stored in the memory 161.

At S230, potential interactions are identified. The potential interactions are identified so that scores can be generated for one or more combinations of pharmaceutical therapeutic(s) and digital therapeutic(s), and the scores may be based on the type and potential severity of the potential interactions. The potential interactions are identified from the interaction information retrieved or generated at S220. The scores may be quantitative, such as between 0 and 100, or qualitative, such as by using descriptive labels such as low, medium and high.

At S240, if only one type of therapeutic is received, an open ended inquiry is made, such as to identify matches for the one type of therapeutic. If only one type of therapeutic is received, the potential matches are ranked at S245, such as if more than one potential match is identified. The rankings may be provided to specify a digital therapeutic which presents the potential interaction with the optimized potential for interaction with a pharmaceutical therapeutic for the first healthcare goal. The rankings at S245 may be provided as scores for digital therapeutic selection. Alternatively, the rankings at S245 may be provided as clusters for digital therapeutic selection, such as when the scores are computed as descriptive labels of low, medium and high. The selection solution described herein makes use of current pharmaceutical and DTx information for digital applications to calculate various scores or descriptive labels to select and recommend pharmaceutical and application combinations that can be prescribed together.

At S250, if two types of therapeutics are received, a close-ended inquiry is made, such as to determine whether the two types of therapeutics interact. If two types of therapeutics are received, the proposed combination of therapeutics is scored at S255. For example, the central computer 150 may generate a score for each potential interaction between a pharmaceutical therapeutic and a digital therapeutic in reaching a first healthcare goal at S250.

At each of S240 and S250, the method of FIG. 2 includes determining that one or more potential interaction exists between therapeutics such as a pharmaceutical therapeutic and a digital therapeutic with respect to one or more healthcare goal. The determinations at S240 and S250 are based on the interaction information for the healthcare goal(s).

At S260, after either S245 or S255, results are output. For example, the central computer 150 may send a result back to the first clinician communication device 111C, the second clinician communication device 112C or the third clinician communication device 113C. The results may be output as feedback, such as to the clinician communication devices. The feedback may include information such as scores as well as selectable options so that a clinician is enabled to make an informed selection as to a combination of therapeutics to prescribe.

Although not shown in FIG. 2, a third type of inquiry may be made when no type of therapeutic is received. For this third type of inquiry, the central computer 150 may determine each type of an appropriate combination of two or more types of therapeutics based on the received diagnosis.

Additionally, the system 100 may provide management for potential interactions once optimal combinations of therapeutics are selected. The management solution may be provided after S260, and may involve monitoring the effects of application usage on pharmaceutical pharmacokinetic (PK) and pharmacodynamic (PD) properties and effects of pharmaceutical usage on application usage. Monitoring information may be used to quantify the inter-therapeutics effects, detecting any potential positive interactions or potential negative interactions that may necessitate changing application or pharmaceutical usage. The central computer 150 may receive information of an update to a digital therapeutic for a healthcare goal, and update the interaction information for the healthcare goal based on the update to the digital therapeutic. For example, the central computer 150 may update interaction information for healthcare goals based on the information of interactions between pharmaceutical therapeutics and digital therapeutics or based on the information of interactions between two different digital therapeutics.

The management results may be fed back to the system 100 to dynamically update the potential characteristic effects generated at S220 and/or the identification of potential interactions at S230. Patients with comorbidities may be monitored for two or more medical conditions. The aspects related to these conditions may be represented as patient and clinician defined goals, and these goals may be addressed by combinations of pharmaceutical therapeutics and digital therapeutics. Different healthcare goals may be weighted in the processing by the central computer 150 in FIG. 1, such as to prioritize one healthcare goal ahead of another healthcare goal. The updates of potential characteristic effects may be used to update feedback and scoring provided to clinicians.

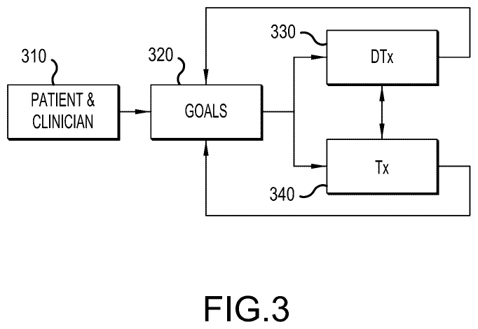

FIG. 3 illustrates a workflow for optimizing therapeutical interactions, in accordance with a representative embodiment.

While managing potential interactions after different types of therapeutics are selected for a patient, the interactions between the different therapeutics may be monitored so that usage of therapeutics or goal definitions is adapted, and this is represented in FIG. 3. As shown, the patient and clinician establish initial goals at 310. The goals are applied initially at 320 to identify and select one or more digital therapeutic at 330 and a pharmaceutical therapeutic at 340. The results of using the digital therapeutic and the pharmaceutical therapeutic are then fed back to dynamically update the goals at 320. The feedback may be used in some circumstances to update the therapeutics prescribed to a patient. The system 100 in FIG. 1 may check the interaction from a DTx and a pharmaceutical therapeutic, but may also take into consideration multiple healthcare goals. When multiple healthcare goals are present for a patient, interaction may be minimized or optimized. Optimization may balance a first healthcare goal and a second healthcare goal, including different adjacent goals for which different combinations of DTx and pharmaceutical therapeutic may present interactions.

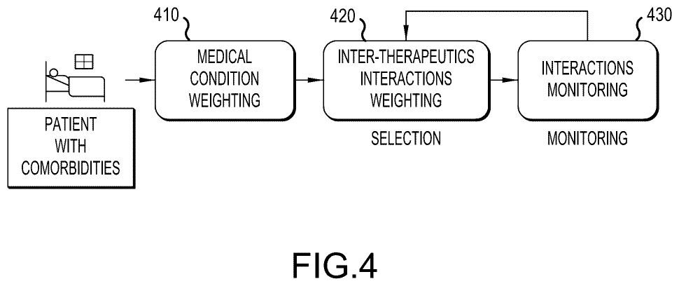

FIG. 4 illustrates another workflow for optimizing therapeutical interactions, in accordance with a representative embodiment.

The selecting and monitoring steps for the therapeutics may be based on computing scores using the central computer 150. The central computer 150 may weight different medical conditions and may also weight the known and detected but previously unknown interactions. The weighting is shown in FIG. 4. As shown, a patient with comorbidities has medical conditions weighted initially at 410. The interaction of therapeutics is initially weighted at 420 when the therapeutics are selected, and the monitored at 430. The monitoring at 430 may result in updating of the interactions at 420 as shown in FIG. 4. The initial weighting at 410 uses known medical conditions and therapeutic interactions to calculate scores to select the best matching therapeutics for prescription at 420. Monitor therapeutics effects at 430, known and previously unknown interactions may be determined and used to update the inter-therapeutics interactions scores.

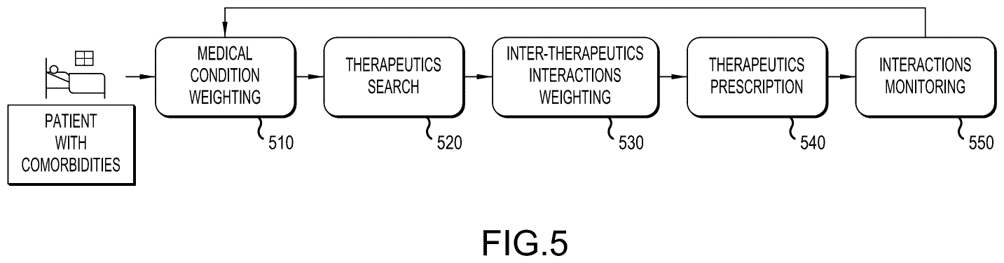

FIG. 5 illustrates another workflow for optimizing therapeutical interactions, in accordance with a representative embodiment.

FIG. 5 includes basic steps for scoring inter-therapeutics interactions and using these for prescription and monitoring decisions. FIG. 5 is an expansion of FIG. 4 to show intermediate steps of search and prescription. The medical condition weighting at 510, the inter-therapeutic interaction weighting at 530 and the interactions monitoring at 550 correspond to the elements shown in FIG. 4. The therapeutics search at 520 and the therapeutics prescription at 540 are added relative to FIG. 4.

The weighting of different medical conditions at 510 is based on the significance (importance) of these conditions from the clinician or patient perspective. The therapeutics search at 520 involves searching for applications and pharmaceuticals that can address the selected medical conditions. The search at 520 may be performed using the descriptive information of the therapeutics. In addition, if there is access to data generated from previous usage of these therapeutics, such as clinical trial results, research results, or other therapeutic usage related data, computational algorithms may be used to perform the search. In one example, the data from the current patient will be compared to data from other patients who have used or tested the corresponding therapeutic. Similar patients may be selected, and effects or interactions related to the selected group of patients may be used for the search.

In another example, therapeutics data and patient data may be used as input to a machine learning algorithm to determine the matching therapeutics. Such a machine learning algorithm may be trained with patient data and therapeutics data as input, and effects, efficacy, PK, PD, adherence, or side effects related aspects as output. Accordingly, the search at 520 may be based on descriptors but it can be also based on performing computations using data related to the therapeutics usage. The inter-therapeutic interaction weighting at 530 uses specific pharmaceutical and application properties to estimate occurrence of potential interactions, whether positive, negative, or unknown. The pharmaceutical and application properties may be used to calculate associated scores. Example pharmaceutical features include different PK properties and side effects. Example application features include application usage frequency, content, and user interface characteristics such as layout, colors, images and/or fonts. Calculated interaction scores may be combined, and a single score representing different therapeutics combinations is calculated. The different therapeutic combinations may include pharmaceutical-pharmaceutical combinations, pharmaceutical-application combinations, and application-application properties. Using these scores, clinicians and patients can rank the pairwise combinations and prescriptions can be provided at 540. Therefore, while the teachings herein focus on pharmaceutical-application interactions or application-pharmaceutical applications, the system 100 may be used for other combinations of therapeutics including therapeutics of the same type. After prescription at 540, therapeutics usage and inter-therapeutics interactions are monitored at 550, resulting in updated interaction scores, and eventually new search at 520 and prescription of new therapeutics at 540 based on updated inter-therapeutic interactions weighting at 530.

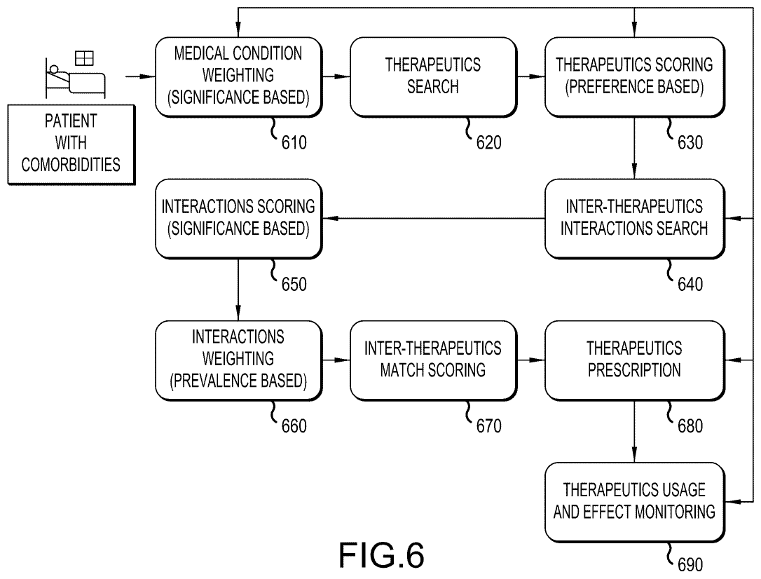

FIG. 6 illustrates another workflow for optimizing therapeutical interactions, in accordance with a representative embodiment.

FIG. 6 is used to describe implementation of score calculations to enable comparison and ranking between different combinations of applications and pharmaceutical options to treat related and sometimes competing medical goals. The workflow in FIG. 6 is particularly relevant to patients with comorbidities and their clinicians.

At 610, medical conditions are weighted. Patients with comorbidities have at least two medical conditions simultaneously present. Typically, one of these conditions is primary and the other(s) are secondary. Some resources define comorbidities as chronic disorders that are not primary reason for hospitalization but which influence hospitalizations. A more flexible definition is used for optimizing therapeutical interactions as described herein, referring to a second primary condition also as comorbidity whenever applicable for generalization. The most common comorbidities that are present in patients discharged from hospitals, which may influence the course of recovery or hospitalization are hypertension (35%), fluid and electrolyte disorders (16%), chronic pulmonary disease (14%), diabetes, uncomplicated (14%), deficiency anemias (11%), renal failure (7%), hypothyroidism (7%), depression (7%), congestive heart failure, and obesity (6%). For most cases, one of the medical conditions is the main reason for the current complaint or hospitalization. From the physician's perspective, this condition is the most important one to treat now in relation to other conditions.

Each condition is assigned a weight at 610, where weights for all relevant conditions sum to one. The condition with the highest significance has the highest weight. The significance definition may vary depending on context, as significance can be defined based on physician or patient preferences, as well short or longer time perspectives. Different aspects may be considered in different implementations to define significance. In one implementation, physician preferences and hospitalization records are followed to determine the weights. Population hospitalization data for patients with similar conditions and similar demographics may be used to estimate future hospitalization reasons and their likelihoods. Taking these into account, the principal reason for the current hospitalization is assigned the highest weight, and weight(s) for the other condition(s) are calculated in line with their likelihoods. The physician may control these calculations and can adjust the final weights. As an example, for two conditions (condition1 and condition2), example weights can be w1=0.7 and w2=0.3, where w1+w2=1.

The therapeutics search at 620 uses a database of pharmaceuticals and applications, to determine solutions to treat condition1 and condition2. Such databases may include descriptive information linking solutions, conditions and treatments. Pre-determined search criteria may be executed, and solutions that address condition1 and condition2 are found. The pre-determined search criteria may include definitions for condition1 and condition2. The result of the therapeutics search at 620 may be two lists, one for condition1 and another for condition2. For example, condition1_list={app1, app2, pharmaceutical1, pharmaceutical2} and condition2_list={app1, app3, pharmaceutical2, pharmaceutical3}. The two lists may include both pharmaceuticals therapeutics and application therapeutics. At the initial performance of 620, neither the interaction between conditions nor the interactions between solutions is considered.

Therapeutics scoring at 630 may involve scoring identified candidates according to the physician and patient preferences. For each candidate, a score between 0 and 100 may be assigned, with 100 indicating the highest preference. For example, for condition1_list_score={(app1, 80), (app2,80), (pharmaceutical1,50), (pharmaceutical2, 30)} and condition2_list_score={(app1, 50), (app3,70), (pharmaceutical2,60), (pharmaceutical3,60)}. The initial preference scores can be calculated at 630 based on historical data of the current physician and patients similar to the current patient, whereas updated preference scores can be later adapted based on patient preferences. For example, quality of life related preferences may be taken into account for updated preference scoring at 630. Initial preference scores may also take into consideration online review notes for applications and pharmaceuticals which were used to determine the initial or other previous preference scores. Final preference scores may also be determined solely on physician and patient discussions and preferences without any initial scores.

In some embodiments, preference driven weights may be assigned for 630, such as for a patient or physician who prefer(s) to use a solution that relates to both conditions, and this may be implemented as a rule: For example, for a pharmaceutical that may be present in both lists weight may be w=1/(# of list entries)+0.2, and for an a application that may be present in both lists weight may be w=1/(# of list entries)+0.1. In this example, when initially different options have the same weight, this will result in condition1_list_score_weights={(app1,0.35), (app2,0.25), (pharmaceutical1,0.25), (pharmaceutical2, 0.45)}, and after normalization condition1_list_score_weights={(app1,0.27), (app2,0.19), (pharmaceutical1,0.19), (pharmaceutical2, 0.35)}. Applying the weights, the preference scores can be updated, by multiplying the preference weight and preference score.

At 640, an inter-therapeutics interactions search is performed. The goal at 640 is to find two solutions where the negative or unknown interactions are minimal, and positive interactions are predictable and controllable. In some circumstances, the goal at 640 may be to find solutions with no interactions to be able to use the solutions as intended. Pairs are constructed by matching solutions from condition1_list and condition2_list, and all possible pairs are considered. Lists of all known or expected interactions may be made based on the search at 640. For example, eight different interaction lists may be prepared: 4 for solution1-solution2, and 4 for solution2-solution1. The four group types may comprise frequent positive interactions, frequent negative interactions, infrequent positive interactions, and infrequent negative interactions. For solution1-solution2, positive interactions may be defined as effects of solution2 that have positive effect on solution1 when solution1 is used to treat condition1. Negative interactions may be defined as effects of solution2 that have negative effects on solution1.

As one example, the effects of pharmaceutical3 on app2 may be the following: app2_pharmaceutical3_common_positive={A, B, C}, app2_pharmaceutical3_common_negative={D, E, F}, app2_pharmaceutical3_uncommon_positive={X, Y}, app2_pharmaceutical3_uncommon_negative={Z, T}.

Four similar lists may be made considering the effects of app2 on pharmaceutical3, when pharmaceutical3 is used to treat condition2. The interactions can be searched using historical data of similar pharmaceuticals and apps. The observed interactions and the expected interactions may be included. The expected interactions may comprise interactions identified based on physician experience for example. Interactions scoring at 650 may be based on significance. The interactions list from 640 may be used to score the interactions in terms of their significance. The scoring at 650 may be particularly important for negative interactions that may otherwise harm the patient. A score between 0 and 100 may be assigned to each interaction, where 100 indicates a significant effect on the patient, and 0 indicates no effect on the patient. For instance, if a certain app feature such as nutrition suggestion is expected to increase toxicity of pharmaceutical it can be marked as 100.

The scores marked at 650 may be added to the corresponding list. For example, app2_pharmaceutical3_common_negative_score={(D,100), (E,20), (F,60)}. The scores may be determined based on prior patient data or based on data from similar patients. In addition, simulation models for the two conditions and effects may be used. Alternatively, a physician may assign the scores.

At 660, interactions weighting is prevalence based. Weights per interaction may be assigned according to observed or expected occurrence of the interaction for the current patient. Data from similar patients may be used at 660. If such data is not available, all interactions may be assigned the same weight.

At 660, the weight per list will sum to 1. For example, app2_pharmaceutical3_common_negative_score_weights={(D,100, prev_w_D), (E,20,prev_w_E), (F,60, prev_w_F)}, where prev_w_d+prev_w_e+prew_w_f=1.

At 670, inter-therapeutics matches are scored. Match scores may be calculated at 670 for all pairwise combinations of different solutions using the scores and weights from previous steps. For the app2_pharmaceutical3 combination the score is calculated by subtracting the weighted score of the negative events from the weighted score of the positive events where alpha and beta are scaling factors between 0 and 1. The calculation of the score is performed as follows:

app2_pharmaceutical3_interaction_score=app2_pharmaceutical3_common_positive_effects+alpha*app2_pharmaceutical3_uncommon_positive_effects−app2_pharmaceutical3_common_negative_effects−beta*app2_pharmaceutical3_uncommon_negative_effect.

The calculation of the weighting is performed as follows:

app2_pharmaceutical3_common_positive_effects=prev_w_A*score_A+prev_w_B*score_B+prev_w_C*score_C

app2_pharmaceutical3_uncommon_positive_effects=prev_w_X*score_X+prev_w_Y*score_Y

app2_pharmaceutical3_common_negative_effects=prev_w_D*score_D+prev_w_E*score_E+prev_w_F*score_F

app2_pharmaceutical3_uncommon_negative_effect=prev_w_Z*score_Z+prev_w_T*score_T

Following the same steps a similar score is calculated based on the effects of app2 on pharmaceutical3, i.e., the pharmaceutical3_app2_interaction_score. The two interaction scores are then combined to calculate a single score for the simultaneous usage of pharmaceutical3 and app2. The single score is calculated as follows:

app2_pharmaceutical3_match_score=w1*(theta*app2_score+app2_pharmaceutical3_interaction_score)+w2*(gamma*pharmaceutical3_score+pharmaceutical3_app2_interaction_score),

where theta and gamma are scaling factors between 0 and 1.

Therapeutics are prescribed at 680. All combinations of solutions for condition1 and condition2 are shown in sorted order based on the solution1_solution2_match_scores. The physician may involve the patient at 680 to use the sorted list as a reference and select the corresponding solutions for prescription.

Therapeutics are used and the effects monitored at 690. The monitoring has two aspects: monitoring related to the interactions identified before the usage and monitoring related to effects on the patient. Data required for quantification of the interactions in terms of prevalence and effect on the patient may be collected. The interaction scores and interaction weights are updated based on the quantification. The type of data that is to be collected and analyzed is determined based on the interaction descriptions. Additionally, effects of simultaneously using the two solutions on a patient is monitored so that new interactions can be discovered. New interactions are added to list, and scores and weights are assigned for the new interactions.

The match score computation in FIG. 6 is repeated with new interaction scores and weights, which can result in new therapeutics prescriptions. Additionally, the monitoring data from 690 may be used to re-evaluate the weights per medical condition and therapeutics preference scores. Adaptations of these may result in changes to prescriptions.

In some examples, the combinations of pharmaceutical therapeutic and digital therapeutic can be chosen so that the pharmaceutical therapeutics focus on treatment of cancer symptoms for certain group of cancer patients with comorbidities. The digital therapeutics may be chosen from the applications that facilitate and enable better management of medication intake related to comorbidities.

In other examples, a digital therapeutic that focuses on mental health management may be selected when the digital therapeutic can improve the overall treatment which requires the use of a certain pharmaceutical therapeutic.

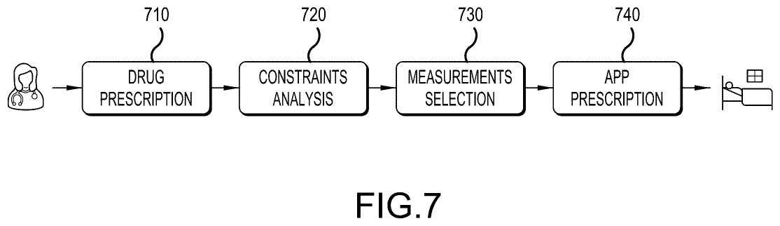

In some embodiments, DTx may be selected to support usage of pharmaceuticals with a narrow therapeutic interval. When narrow therapeutic interval pharmaceuticals are dosed incorrectly serious consequences can occur. For instance, food and exercise can have a major impact on the amount of pharmaceutical in a patient's body. The system 100 may be used to identify appropriate digital applications to support usage of such pharmaceuticals. The basic elements for embodiments such as this are shown in FIG. 7.

FIG. 7 illustrates another workflow for optimizing therapeutical interactions, in accordance with a representative embodiment.

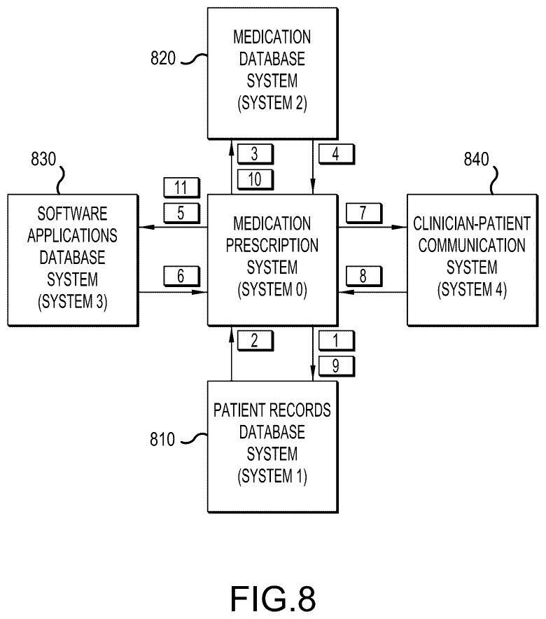

FIG. 7 shows a method for software application selection. The application selection process starts at 710 with collecting and using pharmaceutical prescription information. The constraints linked to the pharmaceutical effects on the particular individual (patient) are determined at 720. These constraints are linked to interaction between the pharmacokinetics (PK) and pharmacodynamics (PD) effects of prescribed pharmaceuticals and patient lifestyle such as exercise, nutrition, and circadian rhythm. Based on the identified and selected constraints, measurements and measurements capabilities are determined and selected at 730. The measurements and measurements capabilities are used to search, select, and prescribe digital solutions such as smart phone applications that can be used to track and improve parameters related to the pharmaceutical PK at 740, and these selections are provided to the clinician and/or the patient. At a high level, FIG. 7 involves determining patient specific pharmaceutical constraint codes, and using the pharmaceutical constraint codes to label applications. The implementation is based on interaction of different systems, and this interaction of different systems is shown in FIG. 8 and described below.

FIG. 8 illustrates a system flow for optimizing therapeutical interactions, in accordance with a representative embodiment.

The essential building logic in FIG. 8 involves having a hospital medication information system that collects and uses current patient, medication, and software application feature information to define, assign, and monitor application labels. The systems in FIG. 8 include a medication prescription system (unlabeled) at the center, a patient records database system 810 on the bottom, a medication database system 820 on the top, a software applications database system 830 on the left, and a clinician-patient communication system 840 on the right. The hospital medication information and prescription system in FIG. 8 is used to manage application prescriptions. The main system in FIG. 8 is the medication prescription system at the center. The medication prescription system interacts with the patient records database system 810, the medication database system 820, the software applications database system 830, and the clinician-patient communication system 840. An example data flow is as follows.

A pharmaceutical is selected, and then specific patient data is requested at 1. Access to the requested patient data is granted at 2. Next, specific medication data is requested at 3, based on the received patient data from 2. Access to the requested medication data is granted at 4. Then based on the received medication data, new patient data can be requested by the medication prescription system. Based on the new patient data, new medication data can be requested. The medication prescription system then processes medication and patient data and pharmaceutical constraints codes (i.e., labels) specific to the determined medication and patient data. At 5, the calculated constraint codes are communicated to the software applications database system 830, and the software applications database system 830 determines feature matches per software based on the constraint codes. The software applications database system 830 communicates per software and per feature the calculated match values at 6. The medication prescription system then ranks the software applications and selects a software application based on the rankings. The selected software is prescribed to the patient at 7 and the communication (data-transfer) settings are also set. At 8, selected patient and software data collected during use is communicated to the medication prescription system. At 9, additional patient data may be requested based on the application usage monitoring. Additional medication data may be requested at 10 based on the application usage monitoring. Additional application coding requirements may be requested at 11 based on the application usage monitoring. The flows in FIG. 8 may be repeated throughout the duration of the prescriptions to any particular patient.

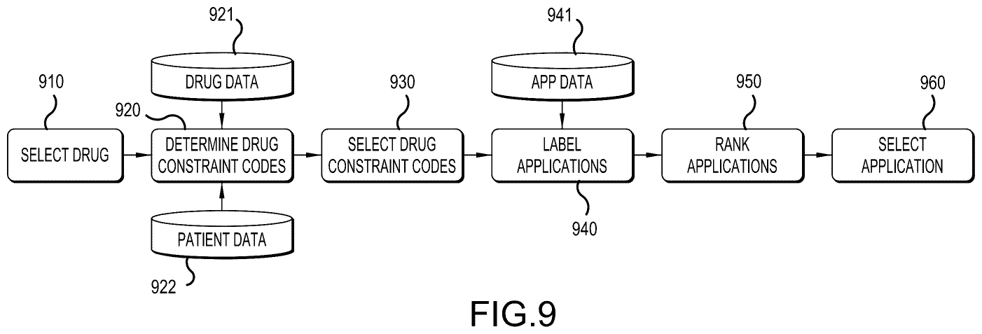

FIG. 9 illustrates another workflow for optimizing therapeutical interactions, in accordance with a representative embodiment.

FIG. 9 illustrates the main processing steps performed by the medication prescription system (system 0) in FIG. 8. First a pharmaceutical is selected at 910. Then, using patient data 922 and pharmaceutical data 921, constraint codes applicable to the selected pharmaceutical are determined at 920. Target constraints codes specific to the patient are selected at 930 and used to assign labels to target applications at 940 using application data 941 After ranking at 950, the application to accompany the pharmaceutical is selected at 960.

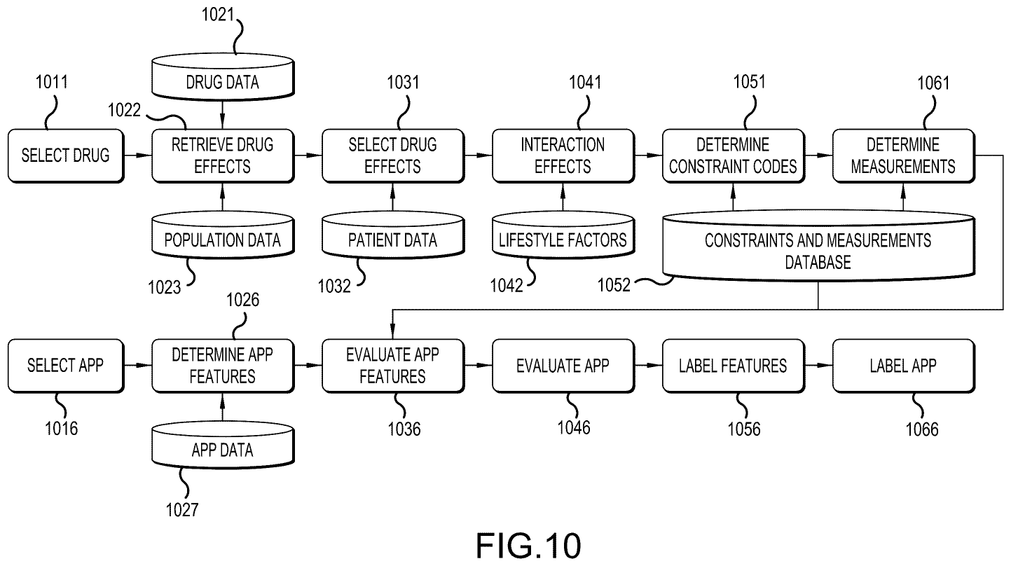

FIG. 10 illustrates another workflow for optimizing therapeutical interactions, in accordance with a representative embodiment.

In some embodiments the constraint codes and application feature are evaluated for labelling as shown in FIG. 10.

A prescription pharmaceutical candidate is selected at 1011. This selection is performed by a clinician using the hospital medication prescription system (system 0) in FIG. 7.

The publicly available pharmaceutical information is retrieved by the medication prescription system at 1022. The retrieved pharmaceutical information includes drug data 1021 including any of pharmacokinetics and pharmacodynamics parameters, side effects, active ingredients, anatomic therapeutic chemical (ATC) classification codes, etc. The pharmaceutical data also includes data collected during the pharmaceutical trial. Additionally, population data 1023 may be accessed at 1022. Population data 1023 includes information from people that have been using the pharmaceutical, and may include side effects, pharmaceutical-pharmaceutical interactions, food-pharmaceutical interactions and measurements and observations related to the effects of the pharmaceutical. The population data may be accessed via health organizations that keep track of pharmaceutical effects, or via a web-based platform. The use of population data 1023 is not particularly required in some embodiments, meaning that the following steps can be completed even when it is not available.

The outputs of 1022 are two lists, list1=(effect1, effect2, effect3) and list2=(effect4, effect5, effect6), for the intended and side effects, respectively.

The next step is determining at 1031 which of the intended pharmaceutical effects and pharmaceutical side effect can apply to the patient. In 1022 the list of intended effects and unwanted side effects of the pharmaceutical were determined. At 1031, the patient data from 1032 is used to determine if the effects are expected to apply to the current patient. The estimation at 1031 is done based on demographics of the patient, on patient history, learnings from the similar patients identified from population data and from people that participated in the pharmaceutical trials. Additionally, clinician input can be used at 1031.

The two outputs of 1031 are dictionaries of wanted and unwanted effects generated by combining the input lists with indicator showing the relevance of effects for the patient. For example, dictionary1={(effect1, 1), (effect2, 0), (effect3,1)}, and dictionary2={(effect4,1), (effect5,0), (effect6,0)}, where dictionary1 and dictionary2 indicate intended effects and unwanted side effects, respectively and the number after the effect is a binary indicator showing the relevance for the patient. Naturally, higher dimensional discrete or continuous indicators can be also used, for instance if there is sufficient data to allow their accurate calculation.

Next, at 1041 expected potential interactions are estimated for the effects that are relevant to the patient (i.e., effect1, effect3 and effect4). Inputs to 1041 are list of interactions learned from the pharmaceutical trials, from populations data, and from PK and PD features of the pharmaceutical. The following interaction categories are prioritized: exercise-pharmaceutical interactions, nutrition-pharmaceutical interactions, and circadian rhythm (exposure to sun light, and sleep patterns)-pharmaceutical interactions. For each of these lifestyle factors from 1042, subcategories can be determined. For example, exercise subcategories can include intensity, frequency, and body location; nutrition subcategories can include consumed food categories that are known to have interaction with the pharmaceutical, as well as food consumption times, and consumption amount; circadian rhythm (CR) categories can include sleep start, end, and duration, and times and duration of exposure to sun light. The result is a list of expected interaction. For example, list3=(exercise_sub1, exercise_sub2, nutrition_sub1, nutrition_sub2, cr_sub1, cr_sub2).

The information about interactions (list3) is used to augment lists of effects (i.e., list1 and list2) with indicators related to the expected patient specific interactions, for example, as shown in Table 1. Data in the table can be represented as matrix [Esub] of size (#effecs)×(#lifestyle factors). Instead of subcategories, a combined matrix [E], of size (#effecs)×3 can be also used to show interactions with main categories exercise, nutrition, and circadian rhythm, as shown in Table 2.

Table 1 below shows a matrix (Esub) showing the potential patient specific interaction between lifestyle sub-factors and pharmaceutical effects:

| | | | | | | |
|---|---|---|---|---|---|---|
| ceutical | e_sub1 | e_sub2 | n_sub1 | n_sub2 | 1 | 2 |
| effects / | | | | | | |
| lifestyle | | | | | | |
| factors | | | | | | |

Table 2 below shows a matrix (E) showing the potential patient specific interaction between lifestyle factors and pharmaceutical effects:

| [symbol] ceutical effects/lifestyle | | |
|---|---|---|
| factors | [symbol] e | [symbol] n |
| [symbol] | [symbol] | [symbol] |

[symbol]

indicates data missing or illegible when filed

Some lifestyle factors may be identified as more important to monitor with respect to pharmaceutical effects and interactions that others based on the pharmaceutical and population information. This information can be captured as a weighting vectors w=[w1, w2, w3] where w1, w2, w3 show the weight for exercise, nutrition, and circadian rhythm, respectively. If the weights are not available, equal weights can be assumed. Using the weight information, a vector of weights w_e=[E]·[w] for the effects can be computed and based on these calculations, effects can be ranked.

Next, the information in Table 1 is used to match the effect-subcategory interaction pairs with pre-defined constraint codes. Constraint codes may reflect a standardized representation for the interactions expected between different pharmaceutical effects and other factors, such as different aspects of exercise, nutrition, circadian rhythm, etc. Constraint codes may also be set and standardized by regulatory bodies or by other standardization bodies. Alternatively, the constraint codes may be defined by healthcare organizations themselves). Once, the applicable codes are selected at 1051, the type of measurements, and their minimum requirements can be determined at 1061 again by referencing to the pre-defined constraint and measurement pairings database 1052. Multiple measurement options may be feasible for a given constraint. Determination of required minimum measurement capabilities may be performed in a hybrid manner, based on pre-determined settings and based on analysis of data collected during preliminary trials conducted in the process of approval of digital applications. At 1016, one or more application(s) is/are selected from an application database that can perform the minimum set of measurements determined at 1061. A general application selection process is described as a measurement/effect specific labelling process. Using labels, the matching applications can be selected at 1016.

First, an application, for which the match or non-match is to be evaluated, is selected at 1016. Application features are retrieved from the application database 1027 at 1026. Next, each feature is evaluated at 1036 in terms of its relation to the required measurements/effects. In 1036, numerical values may be calculated. The numerical values may include score per feature. If a feature can perform and satisfy the requirements it is marked by 1 in the labelling at 1056, otherwise by 0. An example output of 1036 is matrix Mfea shown in Table 3. 1056 may perform labeling by taking scores from 1036 as input and calculating a descriptor per feature. When the score as a result of evaluation is below a given threshold (e.g. 0.5), then a feature can be labelled as not useful, and when above a threshold the feature can be labelled as useful.

Table 3 below shows a matrix (Mfea) showing the application feature measurement capabilities:

| | | | |
|---|---|---|---|
| [symbol] tion features/ | | | |
| measurement | [symbol] ement/effect 1 | [symbol] ement/effect 2 | [symbol] ement/effect 3 |

1
2
3

[symbol]

indicates data missing or illegible when filed

Measurement/effect capabilities per application feature are combined and used to generate a single measure per measurement/effect in an evaluation at 1046 to indicate if the application can be used to perform the measurement/effect. In 1046, numerical values may be calculated. The numerical values may include score per application. 0 and 1 are again examples of such scores. 1056 may also perform labeling by taking scores from 1046 as input and calculating a descriptor per application. 1056 may perform labeling by taking scores from 1036 as input and calculating a descriptor per application. When the score as a result of evaluation is below a given threshold (e.g. 0.5), then an application can be labelled as not useful, and when above a threshold the application can be labelled as useful. For example, if at least one application feature is useful and labelled as such at 1056, the application can be labelled at 1066 as capable to perform the measurement/effect. For example, in Table 3, the application evaluation result is Mapp=[1 1 0]. Other application evaluation methods can be also used, for example by first weighting the features according to their maturity or accuracy, and then computing averages per measurement/effect capability. Based on evaluation results application features are labelled at 1066. For instance, using the Mfea data, they are labelled as useful or non-useful, for 1 and 0, respectively. Using the Mapp (and maybe also Mfea) data, the application is labelled at 1066. For example, an application may be labelled useful if all entries of Mapp are 1, and otherwise not useful. Applications are filtered or searched based on their labels from the labelling at 1066. The matching applications and their feature labels are displayed. In addition to text labels, numerical analyses of feature functionalities (e.g., such as Mfea) are also available for prescribing clinicians to access and examine. Furthermore, a system can use the Mfea data to rank different applications.

As an example of sets of subcategories, a use case for type 1 diabetes patient applications may include finding and prescribing an application for people with type 1 diabetes. This use case is described next.

This use case focuses on exercise-related factors important for type 1 diabetes. The use case provides examples of different types of subcategories and corresponding measurement/effect features that should be present in an application that can be prescribed together with type 1 diabetes medications. Such medications may be prescribed to improve insulin sensitivity and glycaemic control).

### Exercise Modality:

Aerobic exercise: walking, cycling, jogging, swimming
Anaerobic exercise: resistance (strength) training using free weights, weight machines, bodyweight, elastic resistance bands, etc.

### Exercise Intensity:

High intensity interval training.
Moderate intensity
Low intensity
Rest
   Body Parts being Exercised
Part of the body that is the target of exercise

### Timing:

Start, end times and duration of different routines
Alterations between different exercise modalities
Alterations between different exercise intensities, intervals
Short (between intervals) and long (between exercise days) resting periods

### Physiological Monitoring:

Blood glucose monitoring before, during, and after the exercise
Blood ketones or urine ketones monitoring before exercise

The aspects listed above for the type 1 diabetes use case are important to measure and monitor. If an application is to be prescribed as a digital therapeutic for these patients, the application should be capable of measuring the aspects listed above. The aspects listed above correspond to application features that are used for evaluating the suitability for an application.

Nutritional monitoring and management may also be critical for type 1 diabetes. The aspects that are important, which may be incorporated as application features, measurements and/or effects, may include.

### Food Type Monitoring:

Carbohydrate intake (balancing insulin dose to carbohydrate intake)
Glycaemic index
Nutritional distribution percentage (e.g., 45-65% carbohydrate, 20-35% fat, 10-35% protein), also taking into account the exercise routine and intensity and goals

### Food/Drink Intake Times and Amount Monitoring

Time of the day.
Distribution over the day
Time relative to exercise start

### Individual Goals.

Lose weight.
Build muscle strength
Higher performance.
Injury prevention or recovery
Better recovery after exercise

### Fluids Intake.

Water.
Sport and energy drinks
Caffeine intake

Digoxin is used to treat heart failure and in particular irregular heartbeats, such as chronic atrial fibrillation. Digoxin works by affecting sodium and potassium inside heart cells, and it helps the heart to maintain normal, steady and strong heartbeat Digoxin is a drug with narrow therapeutic range, such that getting the right dose is important. The desired effects of digoxin are: reduction in irregular heartbeats, increase in heart beat variability, and reduction in very high heart rate instances. The side effects of digoxin are nausea, vomiting, headache, dizziness, loss of appetite, and diarrhea. More serious side effects may include: weakness, mental/mood changes, vision changes (blurred or yellow/green vision), and/or enlarged/tender breast in men.

Digoxin interactions may therefore be considered in the type 1 diabetes use case. Although there are studies indicating that exercise can influence the serum drug concentration for digoxin, the evidence is limited. So it can be assumed that for digoxin usage, there is no need for any special exercise monitoring and that monitoring options for heart failure patients with AF can be followed. Digoxin has interactions with many food types and drugs. For instance, consumption of banana, fiber rich foods, salt substitutes, Senna and St. John's Wort, black licorice, hawthorn berry and Siberian ginseng should be monitored. Circadian rhythm should also be monitored, insofar as a decrease in diastolic blood pressure and increase in systolic blood pressure during overnight sleep is expected. This effect is not expected during daytime.

FIG. 11 illustrates a computer system, on which a method for optimizing therapeutical interactions is implemented, in accordance with another representative embodiment.

Referring to FIG. 11, the computer system 1100 includes a set of software instructions that can be executed to cause the computer system 1100 to perform any of the methods or computer-based functions disclosed herein. The computer system 1100 may operate as a standalone device or may be connected, for example, using a network 1101, to other computer systems or peripheral devices. In embodiments, a computer system 1100 performs logical processing based on digital signals received via an analog-to-digital converter.

In a networked deployment, the computer system 1100 operates in the capacity of a server or as a client user computer in a server-client user network environment, or as a peer computer system in a peer-to-peer (or distributed) network environment. The computer system 1100 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 1100 can be incorporated as or in a device that in turn is in an integrated system that includes additional devices. In an embodiment, the computer system 1100 can be implemented using electronic devices that provide voice, video or data communication. Further, while the computer system 1100 is illustrated in the singular, the term “system” shall also be taken to include any collection of systems or sub-systems that individually or jointly execute a set, or multiple sets, of software instructions to perform one or more computer functions.

As illustrated in FIG. 11, the computer system 1100 includes a processor 1110. The processor 1110 may be considered a representative example of a processor of a controller and executes instructions to implement some or all aspects of methods and processes described herein. The processor 1110 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1110 is an article of manufacture and/or a machine component. The processor 1110 is configured to execute software instructions to perform functions as described in the various embodiments herein. The processor 1110 may be a general-purpose processor or may be part of an application specific integrated circuit (ASIC). The processor 1110 may also be a microprocessor, a microcomputer, a processor chip, a controller, a microcontroller, a digital signal processor (DSP), a state machine, or a programmable logic device. The processor 1110 may also be a logical circuit, including a programmable gate array (PGA), such as a field programmable gate array (FPGA), or another type of circuit that includes discrete gate and/or transistor logic. The processor 1110 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1100 further includes a main memory 1120 and a static memory 1130, where memories in the computer system 1100 communicate with each other and the processor 1110 via a bus 1108. Either or both of the main memory 1120 and the static memory 1130 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1120 and the static memory 1130 are articles of manufacture and/or machine components. The main memory 1120 and the static memory 1130 are computer-readable mediums from which data and executable software instructions can be read by a computer (e.g., the processor 1110). Each of the main memory 1120 and the static memory 1130 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1100 further includes a video display unit 1150, such as a liquid crystal display (LCD), an organic light emitting diode (OLED), a flat panel display, a solid-state display, or a cathode ray tube (CRT), for example. Additionally, the computer system 1100 includes an input device 1160, such as a keyboard/virtual keyboard or touch-sensitive input screen or speech input with speech recognition, and a cursor control device 1170, such as a mouse or touch-sensitive input screen or pad. The computer system 1100 also optionally includes a disk drive unit 1180, a signal generation device 1190, such as a speaker or remote control, and/or a network interface device 1140.

In an embodiment, as depicted in FIG. 11, the disk drive unit 1180 includes a computer-readable medium 1182 in which one or more sets of software instructions 1184 (software) are embedded. The sets of software instructions 1184 are read from the computer-readable medium 1182 to be executed by the processor 1110. Further, the software instructions 1184, when executed by the processor 1110, perform one or more steps of the methods and processes as described herein. In an embodiment, the software instructions 1184 reside all or in part within the main memory 1120, the static memory 1130 and/or the processor 1110 during execution by the computer system 1100. Further, the computer-readable medium 1182 may include software instructions 1184 or receive and execute software instructions 1184 responsive to a propagated signal, so that a device connected to a network 1101 communicates voice, video or data over the network 1101. The software instructions 1184 may be transmitted or received over the network 1101 via the network interface device 1140.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, optimizing therapeutical interactions enables patients to be provided with solutions to minimize interactions between different types of therapeutics ranging from pharmaceutical therapeutics to DTx. The minimization of interactions may be important for patients with comorbidities, or patients using pharmaceuticals with narrow therapeutic intervals, pharmaceuticals with many interactions, or pharmaceuticals with many side effects. As a result, conflicting treatment goals may be managed for patients. The solutions described herein provide for selection and monitoring of pharmaceuticals and instances of DTx, so that negative interactions are minimized and positive interactions are maximized. Different combinations of therapeutics for a condition may be scored based on measured or expected effects and interactions, so that optimal combinations can be selected and then monitored once prescribed.

Although optimizing therapeutical interactions has been described with reference to several exemplary embodiments, it is understood that the words that have been used are words of description and illustration, rather than words of limitation. Changes may be made within the purview of the appended claims, as presently stated and as amended, without departing from the scope and spirit of optimizing therapeutical interactions in its aspects. Although optimizing therapeutical interactions has been described with reference to particular means, materials and embodiments, optimizing therapeutical interactions is not intended to be limited to the particulars disclosed; rather optimizing therapeutical interactions extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72 (b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

What is claimed is:

1. A system for optimizing therapeutical treatments, comprising:
a central computer with a memory that stores instructions; and a processor that executes the instructions, wherein, when executed by the processor, the instructions cause the system to:
receive at least one of an identification of a pharmaceutical therapeutic for reaching a first healthcare goal or an identification of a digital therapeutic for reaching the first healthcare goal;
retrieve interaction information relating to potential for interactions for the first healthcare goal;
determine that a potential interaction exists between the pharmaceutical therapeutic and the digital therapeutic with respect to the first healthcare goal based on the interaction information for the first healthcare goal; and
generate feedback to reach the first healthcare goal.

2. The system of claim 1, wherein the instructions cause the system further to:
receive both of the identification of the pharmaceutical therapeutic for the first healthcare goal and the identification of the digital therapeutic for the first healthcare goal;
determine that the potential interaction exists between the pharmaceutical therapeutic and the digital therapeutic with respect to the first healthcare goal based on the interaction information for the first healthcare goal; and
generate feedback specifying the potential interaction.

3. The system of claim 1, wherein the instructions cause the system further to:
generate a score for the potential interaction between the pharmaceutical therapeutic and the digital therapeutic in reaching the first healthcare goal; and
include the score in the feedback generated to reach the first healthcare goal.

4. The system of claim 1, wherein the instructions cause the system further to:
receive only the identification of the pharmaceutical therapeutic for the first healthcare goal;
determine an identification of a digital therapeutic which presents optimized potential for interaction with the pharmaceutical therapeutic for the first healthcare goal; and
generate feedback specifying the digital therapeutic which presents the potential interaction with the optimized potential for interaction with the pharmaceutical therapeutic for the first healthcare goal.

5. The system of claim 1, wherein the instructions cause the system further to:
receive only the identification of the digital therapeutic for the first healthcare goal;
determine an identification of a pharmaceutical therapeutic which presents optimized potential for interaction with the digital therapeutic for the first healthcare goal; and
generate feedback specifying the pharmaceutical therapeutic which presents the potential interaction with the optimized potential for interaction with the digital therapeutic for the first healthcare goal.

6. The system of claim 1, further comprising: an anonymization computer configured to anonymize data to be received at the central computer and provide the anonymized data to the central computer.

7. The system of claim 1, wherein the at least one of the identification of the pharmaceutical therapeutic or the identification of the digital therapeutic for the first healthcare goal is or are received from a device used by a clinician or a patient proposing a resolution for reaching the first healthcare goal.

8. The system of claim 1, wherein the instructions cause the system further to:
receive information of an update to a digital therapeutic for the first healthcare goal; and
update the interaction information for the first healthcare goal based on the update to the digital therapeutic for the first healthcare goal.

9. The system of claim 1, wherein the instructions cause the system further to:
receive information of interactions between pharmaceutical therapeutics and digital therapeutics; and
update interaction information for healthcare goals based on the information of interactions between pharmaceutical therapeutics and digital therapeutics.

10. The system of claim 1, wherein the instructions cause the system further to:
receive the at least one of the identification of the pharmaceutical therapeutic for the first healthcare goal and a second healthcare goal or an identification of a digital therapeutic for the first healthcare goal and the second healthcare goal;
weight the first healthcare goal and the second healthcare goal;
retrieve interaction information for the first healthcare goal and the second healthcare goal; and
determine that a potential interaction exists between the pharmaceutical therapeutic and the digital therapeutic with respect to the first healthcare goal and the second healthcare goal based on the interaction information for the first healthcare goal and the second healthcare goal.

11. A method for optimizing therapeutical treatments, comprising:
receiving, at a central computer with a memory that stores instructions and a processor that executes the instructions, at least one of an identification of a pharmaceutical therapeutic for a first healthcare goal or an identification of a digital therapeutic for the first healthcare goal;
retrieving, by the central computer, interaction information relating to potential for interactions for the first healthcare goal;
determining that a potential interaction exists between the pharmaceutical therapeutic and the digital therapeutic with respect to the first healthcare goal based on the interaction information for the first healthcare goal; and
generating feedback to reach the first healthcare goal.

12. The method of claim 11, further comprising:
receiving both of the identification of the pharmaceutical therapeutic for the first healthcare goal and the identification of the digital therapeutic for the first healthcare goal;
determining that the potential interaction exists between the pharmaceutical therapeutic and the digital therapeutic with respect to the first healthcare goal based on the interaction information for the first healthcare goal; and
generating feedback specifying potential for interactions.

13. The method of claim 11, further comprising:
generating a score for the potential interaction between the pharmaceutical therapeutic and the digital therapeutic; and
including the score in the feedback generated to reach the first healthcare goal.

14. The method of claim 11, further comprising:
receiving only the identification of the pharmaceutical therapeutic for the first healthcare goal;
determining an identification of a digital therapeutic which presents optimized potential for interaction with the pharmaceutical therapeutic for the first healthcare goal; and
generating feedback specifying the digital therapeutic which presents the potential interaction with the optimized potential for interaction with the pharmaceutical therapeutic for the first healthcare goal.

15. The method of claim 11, further comprising:
receiving only the identification of the digital therapeutic for the first healthcare goal;
determining an identification of a pharmaceutical therapeutic which presents optimized potential for interaction with the digital therapeutic for the first healthcare goal; and
generating feedback specifying the pharmaceutical therapeutic which presents the potential interaction with the optimized potential for interaction with the digital therapeutic for the first healthcare goal.

16. The method of claim 11, further comprising: anonymizing data to be received at the central computer and providing the anonymized data to the central computer.

17. The method of claim 11, wherein the at least one of the identification of the pharmaceutical therapeutic or the identification of the digital therapeutic for the first healthcare goal is or are received from a device used by a clinician or a patient proposing a resolution for reaching the first healthcare goal.

18. The method of claim 11, further comprising:
receiving information of an update to a digital therapeutic for the first healthcare goal; and
updating the interaction information for the first healthcare goal based on the update to the digital therapeutic for the first healthcare goal.

19. The method of claim 11, further comprising:
receiving information of interactions between pharmaceutical therapeutics and digital therapeutics; and
updating interaction information for healthcare goals based on the information of interactions between pharmaceutical therapeutics and digital therapeutics.

20. The method of claim 11, further comprising:
receiving the at least one of the identification of the pharmaceutical therapeutic for the first healthcare goal and a second healthcare goal or an identification of a digital therapeutic for the first healthcare goal and the second healthcare goal;
weighting the first healthcare goal and the second healthcare goal;
retrieving interaction information for the first healthcare goal and the second healthcare goal; and
determining that an interaction exists between the pharmaceutical therapeutic and the digital therapeutic with respect to the first healthcare goal and the second healthcare goal based on the interaction information for the first healthcare goal and the second healthcare goal.
